(19) United States
(12) Patent Application Publication
(10) Pub. No.: US 2022/0338764 A1
(43) Pub. Date: Oct. 27, 2022

(54) METHOD, APPARATUS AND DEVICE FOR OBTAINING BLOOD GLUCOSE MEASUREMENT RESULT

(71) Applicant: BOE Technology Group Co., Ltd., Beijing (CN)

(72) Inventors: Yuan GAO, Beijing (CN); Xun ZHANG, Beijing (CN); Zhou WANG, Beijing (CN); Dongsheng HUANG, Beijing (CN); Yang HAN, Beijing (CN); Li ZHOU, Beijing (CN); Xin LI, Beijing (CN)

(21) Appl. No.: 17/763,658

(22) PCT Filed: May 21, 2021
(86) PCT No.: PCT/CN2021/095289

§ 371 Date: Mar. 25, 2022

(30) Foreign Application Priority Data
May 27, 2020 (CN) 202010463537.7

(51) Int. Cl. A61B 5/145; A61B 5/00; A61B 5/11; G16H 10/60

(52) U.S. Cl. CPC A61B 5/14532; A61B 5/7267; A61B 5/7246; A61B 5/486; A61B 5/74; A61B 5/4809; A61B 5/1118; A61B 5/14503; G16H 10/60; A61B 2560/0242

(57) ABSTRACT

A method, apparatus and device for obtaining a blood glucose measurement result. A neural network model is trained by using the following method, so as to obtain a trained first neural network model: acquiring a first invasive blood glucose measurement result of a tested object (101); forming a group of new training data by means of same and characteristic values of the most recent PPG signals of the tested object (102); training the neural network model with the training data, so as to obtain a trained first neural network model (106); and after a group of new PPG signals is acquired, extracting characteristic values of the new PPG signals, and inputting the characteristic values into the trained first neural network model, so as to obtain a target blood glucose measurement result (107).

### CROSS-REFERENCE TO RELATED APPLICATIONS

The present application is a U.S. National Phase Entry of International Application No. PCT/CN2021/095289 having an international filing date of May 21, 2021, which claims priority to the Chinese patent application No. 202010463537.7, entitled “Method, Apparatus and Device for Obtaining Blood Glucose Measurement Result”, filed to the CNIPA on May 27, 2020. The entire contents of the above-identified applications are hereby incorporated by reference.

### TECHNICAL FIELD

Embodiments of the present disclosure relate, but are not limited, to the field of blood glucose detection technologies, and in particular, to a method, an apparatus, and a device for obtaining a blood glucose detection result.

### BACKGROUND

At present, diabetes is a typical chronic disease that requires long-term and frequent monitoring, which may cause a series of metabolic disorders in a human body, and is known as a second killer of modern diseases. Means of monitoring diabetes may be: a blood glucose concentration may be effective controlled by frequently detecting the blood glucose concentration and accurately and timely adjusting dosage of oral hypoglycemic drugs and insulin in the human body based on the concentration. Blood glucose detection widely used by the public is a manner of (minimally) invasive blood drop or finger blood plus test paper (hereinafter referred to as invasive blood glucose detection), which usually multiple tests every day and an operation is complicated. A Photoplethysmography (PPG) technology is a non-invasive blood glucose detection method, which may be used for detecting a change in blood volume in a human body. In a process of detection, a fingertip of the human body is irradiated with light having a fixed wavelength, and the light is transmitted to a photoelectric receiver after passing through the fingertip of the human body. When a light beam passes through skin and tissues of the fingertip, part of the light will be absorbed by blood, thus, a light signal received by the photoelectric receiver at the other end will be attenuated. Since skin tissues and muscles have certain stability, their absorption may be regarded as invariable in a process of blood circulation, while blood is flowing, and a blood volume changes regularly with beating of a heart. Therefore, a light intensity received by the photoelectric receiver changes in pulsativity with contraction of the heart. If these light signals with pulsatile changes are converted into electrical signals, Photoplethysmography is obtained. A PPG signal received by the photoelectric receiver may reflect a blood glucose concentration, thus, a value of the blood glucose concentration may be calculated by establishing a mathematical model between the blood glucose concentration and the pulse wave, thereby realizing non-invasive continuous detection. However, a non-invasive blood glucose detection method can only achieve blood glucose trend tracking, and cannot provide more accurate blood glucose detection results.

### SUMMARY

The following is a summary for subject matters described herein in detail. The summary is not intended to limit the scope of protection of claims.

According to a first aspect of the present disclosure, there is provided a method for obtaining a blood glucose detection result, including: training a neural network model by using a following method to obtain a trained first neural network model: acquiring a first invasive blood glucose detection result of a detected object; forming a group of new training data from the first invasive blood glucose detection result and characteristic values of a group of Photoplethysmography (PPG) signals of the detected object collected most recently; training the neural network model with the training data to obtain a trained first neural network model; and extracting characteristic values of a group of new PPG signals after acquiring the group of new PPG signals, and inputting the characteristic values into the trained first neural network model to obtain a target blood glucose detection result.

Optionally, after the forming the group of new training data from the first invasive blood glucose detection result and characteristic values of the group of the Photoplethysmography (PPG) signals of the detected object collected most recently, the method further includes: determining a correlation degree between the new training data and multiple groups of training data in a training set of the first neural network model; determining whether there is target training data whose correlation degree with the new training data reaches a correlation degree threshold in the multiple groups of the training data; comparing the first invasive detection result with a second invasive detection result in the target training data if there is the target training data in the multiple groups of the training data, replacing the target training data with the new training data to obtain an updated training set if a difference between the first invasive detection result and the second invasive detection result is greater than a difference threshold; and training the neural network model with training data in the updated training set.

Optionally, the method further includes: adding the new training data into the training set to obtain an updated training set if there is no target training data in the multiple groups of the training data.

Optionally, the method further includes: acquiring samples of multiple groups of blood glucose influencing factors with labels and samples of blood glucose values with labels; training the neural network model with the samples of the multiple groups of blood glucose influencing factors and the samples of the blood glucose values as training data to obtain a trained second neural network model.

Optionally, the method further includes: acquiring blood glucose influencing factors of the detected object and the target blood glucose detection result; and inputting the blood glucose influencing factors of the detected object and the target blood glucose detection result into the second neural network model, and outputting a health coefficient of the detected object.

Optionally, the blood glucose influencing factors include at least one of following: personal basic information of the detected object, a sleep condition of the detected object, an exercise condition of the detected object, and a weather condition on a detection day.

Optionally, the personal basic information of the detected object includes at least one of following: an age, a height, and a weight of the detected object, and whether the detected object smokes.

Optionally, the acquiring the blood glucose influencing factors of the detected object and the target blood glucose detection result includes: receiving the personal basic information in response to an operation of entering the personal basic information by the detected object; acquiring the sleep condition and the exercise condition of the detected object and the weather condition from a terminal device; quantifying the personal basic information, the sleep condition, the exercise condition, and the weather condition to obtain the blood glucose influencing factors; and acquiring the target blood glucose detection result outputted by the first neural network model.

Optionally, the labels include a degree of influence of the personal basic information on a blood glucose detection result of the detected object, and the method further includes: determining high risk factors influencing a blood glucose value of the detected object according to degrees of influence of the blood glucose influencing factors of the detected object on the blood glucose value of the detected object; determining blood glucose improvement measures corresponding to the high risk factors; and outputting the high risk factors and the blood glucose improvement measures.

Optionally, the method further includes: determining a target blood glucose value range corresponding to the target blood glucose detection result after obtaining the target blood glucose detection result, wherein different blood glucose value ranges correspond to different pieces of prompt information; determining target prompt information corresponding to the target blood glucose value range; and outputting the target prompt information.

According to a second aspect of the present disclosure, there is provided an apparatus for obtaining a blood glucose detection result, including: a first acquisition module configured to acquire a first invasive blood glucose detection result of a detected object; a combination module configured to form a group of new training data from the first invasive blood glucose detection result and characteristic values of a group of Photoplethysmography (PPG) signals of the detected object collected most recently; a first training module configured to train a neural network model with the training data in an updated training set to obtain a trained first neural network model; and a first input module configured to extract characteristic values of a group of new PPG signals after acquiring the group of new PPG signals, and input the characteristic values into the trained first neural network model to obtain a target blood glucose detection result.

Optionally, the apparatus further includes: a first determination module configured to determine a correlation degree between the new training data and multiple groups of training data in a training set of the first neural network model; a determination module configured to determine whether there is target training data whose correlation degree with the new training data reaches a correlation degree threshold in the multiple groups of the training data; an update module configured to compare the first invasive detection result with a second invasive detection result in the target training data if there is the target training data in the multiple groups of the training data, and to replace the target training data with the new training data to obtain an updated training set if a difference between the first invasive detection result and the second invasive detection result is greater than a difference threshold.

Optionally, the update module is further configured to add the new training data into the training set to obtain an updated training set if there is no target training data in the multiple groups of the training data.

According to a third aspect of the present disclosure, there is provided an electronic device, including a processor and a memory storing a computer program executable on the processor, and any one of the above methods for obtaining a blood glucose detection result is implemented when the computer program is executed by the processor.

According to a fourth aspect of the present disclosure, there is provided a non-transitory computer-readable storage medium storing computer-executable instructions for performing any one of the above methods for obtaining a blood glucose detection result.

After the accompanying drawings and detailed descriptions are read and understood, other aspects may be understood.

### BRIEF DESCRIPTION OF DRAWINGS

A brief introduction will be made below to the accompanying drawings need to be used in the embodiments or the prior art. The accompanying drawings in the following description are only one or more embodiments of the present disclosure, and for a person skilled in the art, other drawings may also be obtained according to these drawings on a premise of no creative work.

FIG. 1 is a flowchart of a method for obtaining a blood glucose detection result according to an exemplary embodiment of the present disclosure.

FIG. 2 is a block diagram of an apparatus for obtaining a blood glucose detection result according to an exemplary embodiment of the present disclosure.

FIG. 3 is a block diagram of an electronic device according to an exemplary embodiment of the present disclosure.

### DETAILED DESCRIPTION

Unless otherwise defined, technical terms or scientific terms used in one or more embodiments of the present disclosure should have ordinary meanings as understood by those of ordinary skill in the art to which the present disclosure belongs. “First”, “second”, and similar words used in one or more embodiments of the present disclosure do not represent any order, quantity, or importance, but are merely used for distinguishing different components. “Include”, “contain”, or similar words mean that elements or objects appearing before the words cover elements or objects listed after the words and their equivalents, but do not exclude other elements or objects.

One or more embodiments of the present disclosure provide a method for obtaining a blood glucose detection result, including: training a neural network model by using a following method to obtain a trained first neural network model: acquiring a first invasive blood glucose detection result of a detected object; forming a group of new training data from the first invasive blood glucose detection result and characteristic values of a group of Photoplethysmography (PPG) signals of the detected object collected most recently; training the neural network model with the training data to obtain the trained first neural network model; and extracting characteristic values of a group of new PPG signals after acquiring the group of new PPG signals, and inputting the characteristic values into the trained first neural network model to obtain a target blood glucose detection result.

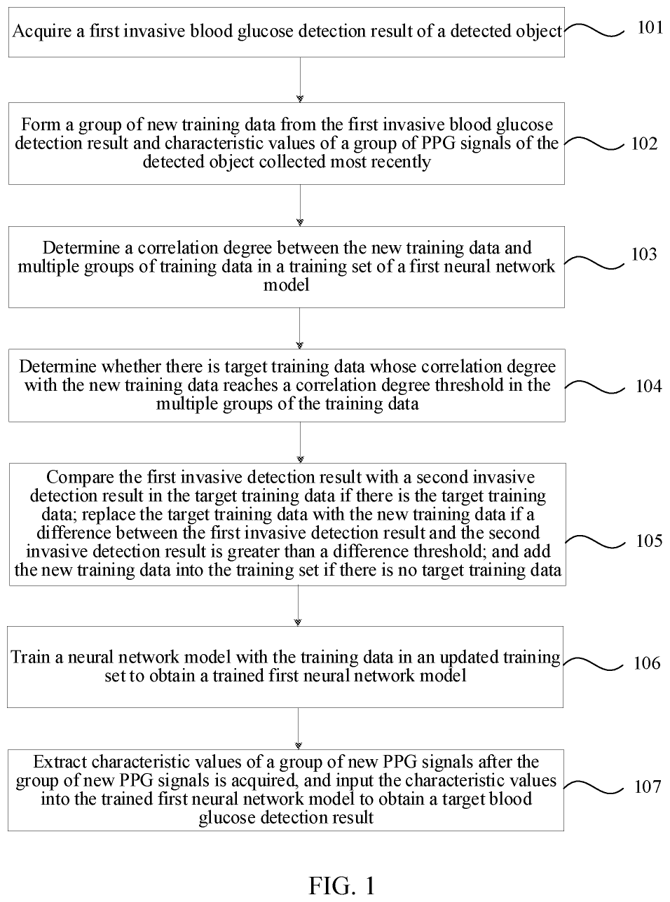

FIG. 1 is a flowchart of a method for obtaining a blood glucose detection result according to an exemplary embodiment of the present disclosure, which may be performed by a terminal device. As shown in FIG. 1, the method includes following acts.

Act 101: acquiring a first invasive blood glucose detection result of a detected object.

For example, the terminal device may establish a Bluetooth or wireless communication connection with a blood glucose detector (for example, a conventional blood glucose detector that performs detection through finger blood) to obtain an invasive blood glucose detection result outputted by the blood glucose detector, which may be, for example, a blood glucose value.

Act 102: forming a group of new training data from the first invasive blood glucose detection result and characteristic values of a group of PPG signals of the detected object collected most recently.

For example, the new training data may be used as a data unit in a training set of a neural network model, with K representing a characteristic value extracted from a PPG signal, M representing a quantity of light sources in the non-invasive blood glucose detector, N representing a quantity of training sets, and C representing an invasive blood glucose detection result, then mathematical expression of this data unit is: [K_{1N}, K_{2N}, K_{3N}, . . . , K_{MN}, C_{N}]^{T}.

Act 103: determining a correlation degree between the new training data and multiple groups of training data in a training set of a first neural network model.

For example, correlation analysis may be performed on the new training data and multiple data units in the training set in sequence, so as to obtain a correlation degree between the new training data and the multiple data units.

The training set of the first neural network model includes multiple data units, and each data unit includes a non-invasive blood glucose detection result and an invasive blood glucose detection result collected in a same period.

Act 104: determining whether there is target training data whose correlation degree with the new training data reaches a correlation degree threshold in the multiple groups of the training data.

The correlation degree threshold may be preset, for example.

Act 105: comparing the first invasive detection result with a second invasive detection result in the target training data if there is the target training data in the multiple groups of the training data; replacing the target training data with the new training data to obtain an updated training set if a difference between the first invasive detection result and the second invasive detection result is greater than a difference threshold; adding the new training data into the training set to obtain an updated training set if there is no target training data in the multiple groups of the training data.

For example, correlation analysis is performed on I_{N}=[K_{1N}, K_{2N}, K_{3N}, . . . , K_{MN}, C_{N}]^{T} and features in first N−1 data units in the training set to determine whether a correlation degree between I_{N}=[K_{1N}, K_{2N}, K_{3N}, . . . , K_{MN}, C_{N}]^{T} and I_{N} reaches 0.8 (an example of the above correlation degree threshold), and if there is no relevant data unit whose correlation degree with I_{N} reaches 0.8, I_{N} is added into the training set; if there is a relevant data unit I_{Q}, it is considered that detection background of I_{N} is consistent with that of I_{Q}, and ∥C_{N}−C_{Q}∥ is calculated, and if a difference is greater than 1 mmol/L (an example of the above difference threshold), it is considered that physiology of the detected object has changed greatly, and at this time, I_{N} is used instead of I_{Q} in the training set, otherwise the training set remains unchanged.

Act 106, training a neural network model with training data in an updated training set to obtain a trained first neural network model; the first neural network model being an Artificial Neural Network (ANN) model, for example.

The above acts 101 to 106 may be performed periodically, so as to adjust the training set according to a physiological condition of the detected object, thereby ensuring accuracy of calculation of the first neural network model.

Act 107: extracting characteristic values of a group of new PPG signals after acquiring the group of new PPG signals, and inputting the characteristic values into the trained first neural network model to obtain a target blood glucose detection result.

After acquiring the group of new PPG signals, the new PPG signals are extracted as test samples that are inputted into the trained first neural network model, so that the target blood glucose detection result outputted by the first neural network model may be obtained. For example, the target blood glucose detection result may be a blood glucose value.

According to the method for obtaining the blood glucose detection result according to one or more embodiments of the present disclosure, a neural network model is trained by using a non-invasive blood glucose detection result and an invasive blood glucose detection result collected in a same period as training data to obtain a trained first neural network model, and a target blood glucose detection result is obtained based on the non-invasive blood glucose detection result by using the model, so that correction of the non-invasive blood glucose detection result may be achieved by using the invasive blood glucose detection result, thereby improving accuracy of the obtained blood glucose detection result. By comparing correlation between a newly added group of training data and other training data in a training set, invalid training data in the training set may also be effectively eliminated to ensure validity of the newly added training data, and accuracy of the blood glucose detection result determined through the first neural network model may be further improved.

In one or more embodiments of the present disclosure, the above method for obtaining the blood glucose detection result may further include: acquiring samples of multiple groups of blood glucose influencing factors with labels and samples of blood glucose values with labels.

For example, different blood glucose influencing factors corresponding to different users and blood glucose values corresponding to the users may be acquired as samples, wherein samples of the different blood glucose influencing factors may have labels with different scores, and samples of different blood glucose values may also have labels with different scores.

The blood glucose influencing factors may include features of a detected object and features that may influence a blood glucose value in features of an environment in which the detected object is located, such as an age, a height, a weight, smoking or not, a sleep condition, and an exercise condition of the detected object as well as a weather condition on a detection day, while the acquired target blood glucose detection result may include a target blood glucose result outputted most recently by the above first neural network model.

The neural network model is trained by using the samples of the multiple groups of blood glucose influencing factors and the samples of the blood glucose values as training data to obtain a trained second neural network model.

Since a non-invasive blood glucose detection device is not easy to wear, or cannot overcome influence of daily use, such as exercise interference, on the detection result, blood glucose monitoring is discontinuous and subject to detection consciousness of a user. Discrete blood glucose values make it difficult to describe a health condition of the user, because there are many blood glucose influencing factors, such as intake of medicine, exercise, diet, weather, sleep, mental mood, obesity, smoking, drinking, and inflammation. Besides recording the blood glucose values, other influencing factors are added to discussion, which is of great significance for individualized blood glucose management.

In an exemplary embodiment of the present disclosure, the method further includes: acquiring blood glucose influencing factors of the detected object and the target blood glucose detection result.

Since these parameters, such as age, height, weight, and smoking or not, are usually relatively stable in a short period of time, and may be entered by the detected object, wherein various parameters used by a system may be expressed as: age: R_{age}=age/10, namely finding a quotient; height: R_{height}=height (cm)/10, namely finding a quotient; weight: R_{weight}=weight (kg), namely finding a whole number; smoking or not: Rₛₘₒₖₑ, which may be scored by the detected object itself according to severity of 0 to 3 points. For example, it may be set as: 0 corresponds to a light degree, 1 point corresponds to a moderate degree, 2 points corresponds to a relatively serious degree, and 3 points corresponds to a serious degree; which is not limited in the embodiment.

Two parameters, sleep and exercise, may be scored automatically by a mobile terminal, wherein the mobile terminal may calculate a quantity of steps that the detected object walks every day through a triaxial acceleration sensor, a gravity sensor, and a triaxial gyroscope that are built-in. Therefore, various parameters used by the system may be expressed as: steps: Rₛₜₑₚ=step/1000; as for sleep, the mobile terminal may be placed on a head of a bed of the detected object to record a quantity of turns of the detected object in one night, for example, sleep: Rₛₗₑₑₚ=sleep/10.

As for a parameter of weather, the mobile terminal may automatically call temperature and humidity data of a current day. For example, the parameter of weather may be expressed as: R_{weather}=Temperature+humidity×100%; wherein, as for a parameter of a target blood glucose detection result, various blood glucose data ranges may be scored in advance by medical experts.

The blood glucose influencing factors of the detected object and the target blood glucose detection result are inputted into the second neural network model, and a health coefficient of the detected object is outputted. The health coefficient may be used for characterizing a health degree of the detected object. For example, a value range of the health coefficient is 0 to 1, and the larger the value of the health coefficient is, the healthier the detected object is.

In one or more embodiments of the present disclosure, the blood glucose influencing factors include at least one of following: personal basic information of the detected object, a sleep condition of the detected object, an exercise condition of the detected object, and a weather condition on a detection day. The blood glucose influencing factors may also include whether the detected object takes a medicine (referring to a medicine that influences a blood glucose value of the detected object), a diet condition of the detected object, mood of the detected object, whether the detected object drinks alcohol, and whether the detected object is currently pregnant or has other diseases, and the like.

In one or more embodiments of the present disclosure, the personal basic information of the detected object includes at least one of following: an age, a height, and a weight of the detected object, and whether the detected object smokes. Optionally, the personal basic information of the detected object may be, for example, information stored in a server entered when the detected object registers the personal basic information, or information stored in the server after the detected object modifies the personal basic information in a subsequent process, and the mobile terminal may acquire the information from the server.

In one or more embodiments of the present disclosure, acquiring the blood glucose influencing factors of the detected object and the target blood glucose detection result may include: receiving the personal basic information in response to an operation of entering the personal basic information by the detected object, for example, entering, by the detected object, his/her personal basic information through the mobile terminal; acquiring one piece or more pieces of following information from the terminal device: a sleep condition, an exercise condition of the detected object, and a weather condition, for example, acquiring the sleep condition of the detected object by calling a sleep management application in the mobile terminal, acquiring the exercise condition of the detected object by calling an exercise management software in the mobile terminal, and acquiring the weather condition of a current day by calling a meteorological application in the mobile terminal; quantifying the personal basic information, the sleep condition, the exercise condition, and the weather condition to obtain the blood glucose influencing factors; and acquiring the target blood glucose detection result outputted by the first neural network model.

In one or more embodiments of the present disclosure, the labels may include a degree of influence of the personal basic information on a blood glucose detection result of the detected object, and the method may further include: determining high risk factors influencing a blood glucose value of the detected object according to the degrees of influence of the blood glucose influencing factors of the detected object on the blood glucose value of the detected object; for example, learning a relationship between a blood glucose value of a user and parameters such as an age, a height, a weight, smoking or not, sleep, and exercise of the user through the second neural network model (for example, an ANN model), so as to obtain high risk factors that lead to increase in the blood glucose value of the detected object.

Blood glucose improvement measures corresponding to the high risk factors are determined. For example, the user may be provided with suggestions on lifestyle habits according to the obtained high risk factors, and if it is determined that the high risk factors leading to the increase in the blood glucose value of the user are lack of sleep and smoking, the user is advised to reduce smoking and go to bed early and get up early.

The high risk factors and the blood glucose improvement measures are output; and the high risk factors and corresponding blood glucose improvement measures are output to the user, so that users may know their own situation and make targeted adjustments.

In one or more embodiments of the present disclosure, the method further includes: determining a target blood glucose value range corresponding to a target blood glucose detection result after obtaining the target blood glucose detection result, wherein different blood glucose value ranges may correspond to different pieces of prompt information, which may be a word, a picture, or a video, and may also include a treatment suggestion; for example, presetting different blood glucose value ranges to correspond to different pieces of prompt information that may include going to a hospital for treatment, self-injection of insulin, or taking another medicine, or maintaining a current situation and the like; and determining target prompt information corresponding to the target blood glucose value range; and outputting the target prompt information. For example, if a blood glucose value range of 4.0 to 6.1 mmol/L is preset to correspond to prompt information of maintaining a current situation, then a target blood glucose detection result is obtained to be 5 mmol/L, a target blood glucose value range is determined to be 4.0 to 6.1 mmol/L, and target prompt information corresponding to the target blood glucose value range is maintaining a current situation for treatment. In addition, when a blood glucose value range corresponding to a detection result of a target blood glucose value of the detected object corresponds to prompt information of going to a hospital for treatment, an alarm function may be started and an alarm message may be sent to prompt the detected object or family members of the detected object, so that treatment measures may be taken in time.

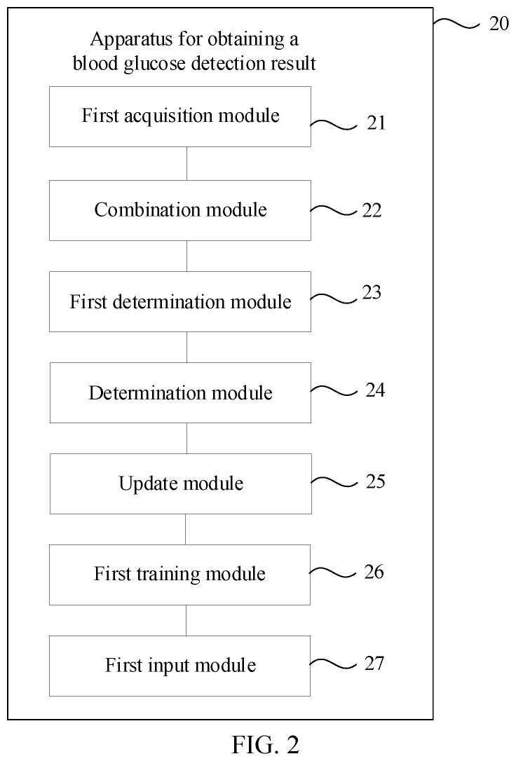

FIG. 2 is a block diagram of an apparatus for obtaining a blood glucose detection result according to an exemplary embodiment of the present disclosure. As shown in FIG. 2, the apparatus 20 includes: a first acquisition module 21 configured to acquire a first invasive blood glucose detection result of a detected object; a combination module 22 configured to form a group of new training data from the first invasive blood glucose detection result and characteristic values of a group of Photoplethysmography (PPG) signals of the detected object collected most recently; a first determination module 23 configured to determine a correlation degree between the new training data and multiple groups of training data in a training set of a first neural network model; a determination module 24 configured to determine whether there is target training data whose correlation degree with the new training data reaches a correlation degree threshold in the multiple groups of the training data; an update module 25 configured to compare the first invasive detection result with a second invasive detection result in the target training data if there is the target training data in the multiple groups of the training data, to replace the target training data with the new training data to obtain an updated training set if a difference between the first invasive detection result and the second invasive detection result is greater than a difference threshold, and to add the new training data into the training set to obtain an updated training set if there is no target training data in the multiple groups of the training data; a first training module 26 configured to train the neural network model with training data in the updated training set to obtain a trained first neural network model; and a first input module 27 configured to extract characteristic values of a group of new PPG signals after the group of new PPG signals is acquired, and input the characteristic values into the trained first neural network model to obtain a target blood glucose detection result.

In one or more embodiments of the present disclosure, the apparatus further includes: a second acquisition module configured to acquire samples of multiple groups of blood glucose influencing factors with labels and samples of blood glucose values with labels; and a second training module configured to train the neural network model by using the samples of the multiple groups of blood glucose influencing factors and the samples of the blood glucose values as the training data to obtain a trained second neural network model.

In one or more embodiments of the present disclosure, the apparatus further includes: a third acquisition module configured to acquire blood glucose influencing factors of the detected object and the target blood glucose detection result; a second input module configured to input the blood glucose influencing factors of the detected object and the target blood glucose detection result into the second neural network model, and output a health coefficient of the detected object.

In one or more embodiments of the present disclosure, the blood glucose influencing factors include at least one of following: personal basic information of the detected object, a sleep condition of the detected object, an exercise condition of the detected object, and a weather condition on a detection day.

In one or more embodiments of the present disclosure, the personal basic information of the detected object includes at least one of following: an age, a height, and a weight of the detected object, and whether the detected object smokes.

In one or more embodiments of the present disclosure, the apparatus may further include a data acquisition module configured to: receive the personal basic information in response to an operation of entering the personal basic information by the detected object; acquire a sleep condition and an exercise condition of the detected object and a weather condition from a terminal device; quantify the personal basic information, the sleep condition, the exercise condition, and the weather condition to obtain the blood glucose influencing factors; and acquire a target blood glucose detection result outputted by the first neural network model.

In one or more embodiments of the present disclosure, the labels include a degree of influence of the personal basic information on a blood glucose detection result of the detected object, and the apparatus further includes: a second determination module configured to determine high risk factors influencing a blood glucose value of the detected object according to a degree of influence of each parameter in the personal basic information on the blood glucose value of the detected object; a third determination module configured to determine blood glucose improvement measures corresponding to the high risk factors; and a first output module configured to output the high risk factors and the blood glucose improvement measures.

In one or more embodiments of the present disclosure, the apparatus further includes: a fourth determination module configured to determine a target blood glucose value range corresponding to the target blood glucose detection result after obtaining the target blood glucose detection result, wherein different blood glucose value ranges correspond to different pieces of prompt information; a fifth determination module configured to determine target prompt information corresponding to the target blood glucose value range; and a second output module configured to output the target prompt information.

One or more embodiments of the present disclosure further provide an electronic device, including a processor and a memory storing a computer program executable on the processor, and any one of the above methods for obtaining a blood glucose detection result is implemented when the computer program is executed by the processor.

One or more embodiments of the present disclosure further provide a non-transitory computer-readable storage medium, storing computer-executable instructions for implementing any one of the above methods for obtaining a blood glucose detection result.

The methods according to one or more embodiments of the present disclosure may be executed by a single device, such as a computer or a server. The methods of the embodiments may also be applied in a distributed scenario, and are completed by cooperation of multiple devices. In such a case of the distributed scenario, one of the multiple devices may only perform one or more acts of the methods according to one or more embodiments of the present disclosure, and the multiple devices interact with each other to complete the methods.

Specific embodiments of the present disclosure are described above. Other embodiments fall within the scope of the appended claims. In some cases, actions or acts recorded in the claims may be performed in an order different from those in the embodiments and may still achieve desired results. In addition, processes illustrated in the drawings do not necessarily require a specific order or continuous order illustrated to achieve the desired results. In some embodiments, multitask processing and parallel processing are feasible or possibly advantageous.

In the present disclosure, for convenience of description, in the description of the above apparatus, functions are respectively described in a form of various modules. Of course, in implementation of one or more embodiments of the present disclosure, functions of various modules may be implemented in a same or multiple software and/or hardware.

The apparatuses in the above embodiments are configured to implement corresponding methods in the above embodiments, and have beneficial effects of corresponding method embodiments, which will not repeated here.

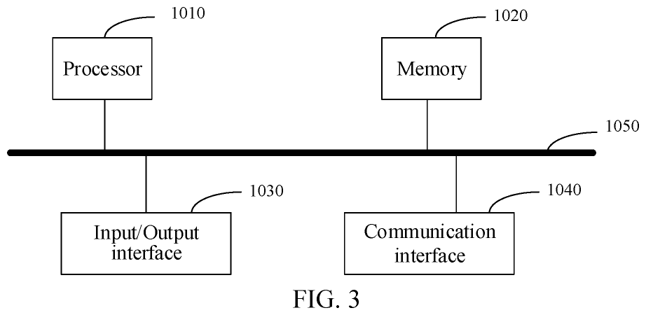

FIG. 3 shows a more specific schematic diagram of a hardware structure of an electronic device according to one or more embodiments of the present disclosure. The device may include a processor 1010, a memory 1020, an input/output interface 1030, a communication interface 1040, and a bus 1050. The processor 1010, the memory 1020, the input/output interface 1030, and the communication interface 1040 achieve communication connections among each other inside the device via the bus 1050.

The processor 1010 may be implemented by means of a General Central Processing Unit (CPU), a microprocessor, an Application Specific Integrated Circuit (ASIC), or one or more integrated circuits, and is configured to execute related programs to implement the technical solutions provided by the embodiments of the present disclosure.

The memory 1020 may be implemented in a form of a Read Only Memory (ROM), a Random Access Memory (RAM), a static storage device, a dynamic storage device, etc. The memory 1020 may store an operating system and other application programs. When the technical solutions provided by the embodiments of the present disclosure are implemented through software or firmware, related program codes are stored in the memory 1020 and called and executed by the processor 1010.

The input/output interface 1030 is configured to be connected with an input/output module to achieve information input and output. The input/output module may be configured in a device as a component (not shown in the figure) or externally connected with a device to provide a corresponding function. An input device may include a keyboard, a mouse, a touch screen, a microphone, various sensors, and the like. An output device may include a display, a speaker, a vibrator, an indicator lamp, and the like.

The communication interface 1040 is configured to be connected with a communication module (not shown in the figure) to implement communication interaction between the device and another device. The communication module may implement communication through a wired mode (such as a Universal Serial Bus (USB) or a network cable) or a wireless mode (such as a mobile network, Wireless Fidelity (WiFi), or Bluetooth).

The bus 1050 includes a path for transmitting information among various components (for example, the processor 1010, the memory 1020, the input/output interface 1030, and the communication interface 1040) of the device.

Although the above device only shows the processor 1010, the memory 1020, the input/output interface 1030, the communication interface 1040, and the bus 1050, in a specific implementation process, the device may further include other components necessary for normal operation. In addition, the above device may only include components necessary for implementing solutions of the embodiments of the present disclosure, rather than all of components shown in the drawings.

A computer-readable medium of the embodiment includes permanent and non-permanent, removable and non-removable media, which may implement information storage by any method or technology. Information may be computer-readable instructions, a data structure, a module of a program, or other data. Examples of storage media of a computer include, but are not limited to, a Phase-change Random Access Memory (PRAM), a Static Random Access Memory (SRAM), a Dynamic Random Access Memory (DRAM), other types of Random Access Memory (RAM), a Read Only Memory (ROM), an Electrically Erasable Programmable Read Only Memory (EEPROM), a flash memory or other memory technology, a Compact Disc-Read Only Memory (CD-ROM), a Digital Versatile Disc (DVD) or other optical storage, a cartridge magnetic tape, a magnetic tape and magnetic disk memory or other magnetic storage device or any other non-transmission medium, which may be used for storing information that may be accessed by a computing device.

Discussions in any of the above embodiments are only exemplary and are not intended to imply that the scope of the present disclosure (including the claims) is limited to these examples. Under a concept of the present disclosure, the above embodiments or technical features in different embodiments may be combined, and the acts may be implemented in any order, and there are many other changes in different aspects of one or more embodiments of the present disclosure as described above, which are not provided in details for brevity.

In addition, well-known power/ground connections with an Integrated Circuit (IC) chip and another component may or may not be shown in the drawings provided in order to simplify description and discussion and not to obscure one or more embodiments of the present disclosure. Moreover, an apparatus may be illustrated in a form of a block diagram in order to avoid obscuring one or more embodiments of the present disclosure, which also considers a following fact, that is, details about implementation modes of apparatuses in these block diagrams highly depend on a platform on which one or more embodiments of the present disclosure will be implemented (that is, these details should be fully within a understanding range of those skilled in the art). In a case where specific details (for example, circuits) are set forth to describe exemplary embodiments of the present disclosure, one or more embodiments of the present disclosure may be implemented without these specific details or in a case where there are changes in these specific details. Therefore, these descriptions should be considered illustrative rather than restrictive.

Although the present disclosure has been described in combination with the embodiments of the present disclosure, many alternatives, modifications, and variations of these embodiments are intended to be included within the scope of protection of the appended claims according to the foregoing description. For example, the discussed embodiments may be used for other memory architectures (e.g., a Dynamic RAM (DRAM)).

One or more embodiments of the present disclosure are intended to cover all such alternatives, modifications, and variations that fall within the broad scope of the appended claims. Therefore, any omissions, modifications, equivalent replacements, improvements, etc., made within the spirit and principle of one or more embodiments of the present disclosure shall be included within the scope of protection of the present disclosure.

1. A method for obtaining a blood glucose detection result, comprising:
training a neural network model by using a following method to obtain a trained first neural network model:
acquiring a first invasive blood glucose detection result of a detected object;
forming a group of new training data from the first invasive blood glucose detection result and characteristic values of a group of Photoplethysmography (PPG) signals of the detected object collected most recently;
training the neural network model with the training data to obtain the trained first neural network model; and
extracting characteristic values of a group of new PPG signals after acquiring the group of new PPG signals, and inputting the characteristic values into the trained first neural network model to obtain a target blood glucose detection result.

2. The method according to claim 1, wherein after the forming the group of new training data from the first invasive blood glucose detection result and the characteristic values of the group of the Photoplethysmography (PPG) signals of the detected object collected most recently, the method further comprises:
determining a correlation degree between the new training data and multiple groups of training data in a training set of the first neural network model;
determining whether there is target training data whose correlation degree with the new training data reaches a correlation degree threshold in the multiple groups of the training data;
comparing the first invasive detection result with a second invasive detection result in the target training data when there is the target training data in the multiple groups of the training data, replacing the target training data with the new training data to obtain an updated training set when a difference between the first invasive detection result and the second invasive detection result is greater than a difference threshold; and
training the neural network model with training data in the updated training set.

3. The method according to claim 2, further comprising: adding the new training data into the training set to obtain an updated training set when there is no target training data in the multiple groups of the training data.

4. The method according to claim 1, further comprising:
acquiring samples of multiple groups of blood glucose influencing factors with labels and samples of blood glucose values with labels; and
training the neural network model with the samples of the multiple groups of blood glucose influencing factors and the samples of the blood glucose values as training data to obtain a trained second neural network model.

5. The method according to claim 4, further comprising:
acquiring blood glucose influencing factors of the detected object and the target blood glucose detection result; and
inputting the blood glucose influencing factors of the detected object and the target blood glucose detection result into the second neural network model, and outputting a health coefficient of the detected object.

6. The method according to claim 4, wherein the blood glucose influencing factors comprise at least one of following:
personal basic information of the detected object, a sleep condition of the detected object, an exercise condition of the detected object, and a weather condition on a detection day.

7. The method according to claim 6, wherein the personal basic information of the detected object comprises at least one of following:
an age, a height, and a weight of the detected object, and whether the detected object smokes.

8. The method according to claim 6, wherein the acquiring the blood glucose influencing factors of the detected object and the target blood glucose detection result comprises:
receiving the personal basic information in response to an operation of entering the personal basic information by the detected object;
acquiring the sleep condition and the exercise condition of the detected object and the weather condition from a terminal device;
quantifying the personal basic information, the sleep condition, the exercise condition, and the weather condition to obtain the blood glucose influencing factors; and
acquiring the target blood glucose detection result outputted by the first neural network model.

9. The method according to claim 6, wherein the labels comprise a degree of influence of the personal basic information on a blood glucose detection result of the detected object, and the method further comprises:
determining high risk factors influencing a blood glucose value of the detected object according to degrees of influence of the blood glucose influencing factors of the detected object on the blood glucose value of the detected object;
determining blood glucose improvement measures corresponding to the high risk factors; and
outputting the high risk factors and the blood glucose improvement measures.

10. The method according to claim 1, further comprising:
determining a target blood glucose value range corresponding to the target blood glucose detection result after obtaining the target blood glucose detection result, wherein different blood glucose value ranges correspond to different pieces of prompt information;
determining target prompt information corresponding to the target blood glucose value range; and
outputting the target prompt information.

11. An apparatus for obtaining a blood glucose detection result, comprising a processor and a memory storing a computer program executable by the processor, wherein the processor is configured to perform following operations when executing the computer program:
acquiring a first invasive blood glucose detection result of a detected object;
forming a group of new training data from the first invasive blood glucose detection result and characteristic values of a group of Photoplethysmography (PPG) signals of the detected object collected most recently;
training a neural network model with the training data to obtain a trained first neural network model; and
extracting characteristic values of a group of new PPG signals after acquiring the group of new PPG signals, and inputting the characteristic values into the trained first neural network model to obtain a target blood glucose detection result.

12. The apparatus according to claim 11, wherein the processor is further configured to perform following operations when executing the computer program:
determining a correlation degree between the new training data and multiple groups of training data in a training set of the first neural network model;
determining whether there is target training data whose correlation degree with the new training data reaches a correlation degree threshold in the multiple groups of the training data; and
comparing the first invasive detection result with a second invasive detection result in the target training data when there is the target training data in the multiple groups of the training data, and replacing the target training data with the new training data to obtain an updated training set when a difference between the first invasive detection result and the second invasive detection result is greater than a difference threshold.

13. The apparatus according to claim 12, wherein the processor is further configured to perform a following operation when executing the computer program: adding the new training data into the training set to obtain an updated training set when there is no target training data in the multiple groups of the training data.

14. (canceled)

15. A non-transitory computer-readable storage medium, storing computer-executable instructions for performing the method according to claim 1.

16. The method according to claim 5, wherein the blood glucose influencing factors comprise at least one of following:
personal basic information of the detected object, a sleep condition of the detected object, an exercise condition of the detected object, and a weather condition on a detection day.

17. The method according to claim 16, wherein the personal basic information of the detected object comprises at least one of following:
an age, a height, and a weight of the detected object, and whether the detected object smokes.

18. The method according to claim 16, wherein the acquiring the blood glucose influencing factors of the detected object and the target blood glucose detection result comprises:
receiving the personal basic information in response to an operation of entering the personal basic information by the detected object;
acquiring the sleep condition and the exercise condition of the detected object and the weather condition from a terminal device;
quantifying the personal basic information, the sleep condition, the exercise condition, and the weather condition to obtain the blood glucose influencing factors; and
acquiring the target blood glucose detection result outputted by the first neural network model.

19. The method according to claim 16, wherein the labels comprise a degree of influence of the personal basic information on a blood glucose detection result of the detected object, and the method further comprises:
determining high risk factors influencing a blood glucose value of the detected object according to degrees of influence of the blood glucose influencing factors of the detected object on the blood glucose value of the detected object;
determining blood glucose improvement measures corresponding to the high risk factors; and
outputting the high risk factors and the blood glucose improvement measures.

20. The method according to claim 2, further comprising:
determining a target blood glucose value range corresponding to the target blood glucose detection result after obtaining the target blood glucose detection result, wherein different blood glucose value ranges correspond to different pieces of prompt information;
determining target prompt information corresponding to the target blood glucose value range; and
outputting the target prompt information.

21. The method according to claim 3, further comprising:
determining a target blood glucose value range corresponding to the target blood glucose detection result after obtaining the target blood glucose detection result, wherein different blood glucose value ranges correspond to different pieces of prompt information;
determining target prompt information corresponding to the target blood glucose value range; and
outputting the target prompt information.
